(19) United States
(12) Patent Application Publication
(10) Pub. No.: US 2012/0310091 A1
(43) Pub. Date: Dec. 6, 2012

(54) ULTRASOUND IMAGE DIAGNOSTIC APPARATUS

(75) Inventors: Kenji OHNUMA, Tokyo (JP); Takayuki SASAKI, Tokyo (JP); Tetsuya TANIGUCHI, Tokyo (JP); Makoto HORIUCHI, Tokyo (JP); Katsumi HOSOKAWA, Tokyo (JP); Kenji SUZUKI, Tokyo (JP); Kikurou NEMOTO, Tokyo (JP); Kazuya OSADA, Tokyo (JP)

(73) Assignee: KONICA MINOLTA MEDICAL & GRAPHIC, INC., Tokyo (JP)

(21) Appl. No.: 13/481,357

(22) Filed: May 25, 2012

(30) Foreign Application Priority Data
May 31, 2011 (JP) JP2011-121438

(51) Int. Cl. A61B 8/15

(52) U.S. Cl. 600/443

(57) ABSTRACT

The transmitting section of an ultrasound image diagnostic apparatus outputs a square pulse signal to allow a transducer to generate transmission ultrasound. The transmitting section sets the duty ratio of a pulse signal so that one of peaks of the first step response of the transducer which is generated when a change is made to voltage of the pulse signal overlaps with one of peaks of the second step response of the transducer which is generated when another change is made to the voltage of the pulse signal.

This application is based on Japanese Patent Application No. 2011-121438 filed on May 31, 2011 with Japan Patent Office, the entire content of which is hereby incorporated by reference.

### BACKGROUND OF THE INVENTION

The present invention relates to an ultrasound image diagnostic apparatus.

In the conventional ultrasound image diagnostic apparatus, using an ultrasound probe, ultrasound (transmission ultrasound) is transmitted to a subject to be examined such as a living body and then received ultrasound (reflected ultrasound) is converted into a reception signal to display an ultrasound image based thereon. Since such reflected ultrasound contains information showing the state of the interior of an examined subject, it is critical to acquire high-quality reflected ultrasound in order to obtain a high-quality ultrasound image. Signal processing for a reception signal makes it possible to enhance the image quality of an ultrasound image. However, basically, it is desirable for transmission ultrasound to have a high quality.

High-quality transmission ultrasound is said to be excellent in spatial resolution and reaching depth (penetration). To realize enhanced spatial resolution, the pulse length of transmission ultrasound is required to be short. In other words, by allowing the frequency band of transmission ultrasound to have broader band or allowing the frequency thereof to increase, a shorter pulse can be realized. On the other hand, to realize excellent reaching depth, it is necessary that ultrasound making a round trip in the interior of an examined subject be resistant to attenuation, and this requirement can be satisfied by the increase of the amplitude of ultrasound or the decrease of the frequency of transmission ultrasound. Namely, it is preferable to output a broadband transmission ultrasound having both of a low-frequency component and a high-frequency component and having a large amplitude and small pulse width.

In view of such situations, in the conventional ultrasound image diagnostic apparatus, there is disclosed an apparatus, in which to realize a desired waveform of transmission ultrasound, using an arbitrary waveform generating system, a drive signal appropriate for the characteristics of a transducer is provided (for example, Japanese Translation of PCT International Application Publication No. 2005-536309).

Further, there is also disclosed an ultrasound image diagnostic apparatus, in which the duty ratio of a drive signal of a square wave is changed for outputting broadband transmission ultrasound (for example, U.S. Pat. No. 5,833,614 specification).

Over recent years, ultrasound probes having broadband characteristics have been known. Such ultrasound probes make it possible to transmit and receive ultrasound in a broadband being, therefore, highly useful.

However, as described in Japanese Translation of PCT International Application Publication No. 2005-536309, since an arbitrary waveform generating system can output transmission ultrasound having a broadband waveform, an ultrasound probe exhibiting broadband characteristics can be efficiently utilized, but the circuit size and the device size increase, resulting in increased cost.

Further, the technique of U.S. Pat. No. 5,833,614 specification makes it possible to output broadband transmission ultrasound, but no characteristics of an ultrasound probe are taken into consideration. Therefore, the characteristics of the ultrasound probe cannot be efficiently utilized.

### SUMMARY

One of the objects of the present invention is to provide an ultrasound image diagnostic apparatus, in which an ultrasound probe exhibiting broadband characteristics is efficiently utilized at reduced cost and thereby a high-quality ultrasound image can be acquired.

For the purpose of realization of at least one of the above objects, an ultrasound image diagnostic apparatus reflecting one aspect of the present invention is described below.

1. An ultrasound image diagnostic apparatus including:

an ultrasound probe which has a transducer for outputting a transmission ultrasound toward a subject to be examined by using a pulse signal and for receiving a reflected ultrasound from the subject so as to output a reception signal; and

a transmitting section for outputting a square pulse signal so as to allow the transducer to generate the transmission ultrasound,

wherein the transmitting section is configured to set a duty ratio of the pulse signal so that one of peaks of a first step response of the transducer overlaps with one of peaks of a second step response of the transducer, the first step response being generated when a change is made to voltage of the pulse signal and the second step response being generated when another change is made to the voltage of the pulse signal.

2. The ultrasound image diagnostic apparatus of the item 1,

wherein the transmitting section is configured to set the duty ratio so that three peaks constituted of one of peaks of a third step response, one of peaks of the second step response and one of peaks of the first step response overlap with one another, the third step response being generated because of voltage change caused after the first and the second step responses.

3. The ultrasound image diagnostic apparatus of the item 1 or 2,

wherein the transmitting section is configured to output the pulse signal so as to ensure that a frequency band characteristic of the transmission ultrasound generated by the transducer is wider than a frequency band characteristic of the transducer.

4. The ultrasound image diagnostic apparatus of any one of the items 1-3,

wherein the transmitting section is configured to be capable of changing the duty ratio.

5. The ultrasound image diagnostic apparatus of any one of the items 1-4,

wherein the transmitting section is configured to change the voltage of the pulse signal so that an amplitude of the pulse signal is identical in positive polarity and negative polarity.

6. The ultrasound image diagnostic apparatus of any one of the items 1-5, further including:

a filter section for filtering a predetermined frequency component in the reception signal; and

a band setting section for changing a frequency component to be filtered by the filter section according to an acquisition depth specified by time of acquisition of the reception signal.

7. The ultrasound image diagnostic apparatus of any one of the items 1-6,

wherein the transducer is configured to have a frequency band characteristic which enables the transducer to receive a baseband component generated due to traveling of the transmission ultrasound in an interior of the subject.

### BRIEF DESCRIPTION OF THE DRAWINGS

FIG. 1 is a view showing an external configuration of an ultrasound image diagnostic apparatus.

FIG. 2 is a block diagram showing a schematic configuration of the ultrasound image diagnostic apparatus.

FIG. 3 is a block diagram showing a schematic configuration of a transmitting section.

FIG. 4 is a block diagram showing a schematic configuration of an image generating section.

FIG. 5 is a view illustrating the drive waveform of a pulse signal;

FIGS. 6a and 6b are views illustrating a step response of a transducer with respect to a pulse application.

FIGS. 7a, 7b, and 7c are views illustrating step responses of a transducer with respect to pulse applications.

FIGS. 8a and 8b are views illustrating a response of a transducer with respect to the drive waveform of a pulse signal.

FIGS. 9a and 9b are views illustrating one example of the response of a transducer with respect to the drive waveform of a pulse signal.

FIGS. 10a and 10b are views showing a spectrum of a conventional transmission ultrasound.

FIGS. 11a and 11b are views showing spectra of the transmission ultrasound and the reflected ultrasound according to the present embodiment

FIG. 12 is a view showing the spectrum of reflected ultrasound obtained by the transmission ultrasound according to another aspect of the present embodiment

### DETAILED DESCRIPTION OF THE PREFERRED EMBODIMENT

The ultrasound image diagnostic apparatus according to the preferred embodiment of the present invention will now be described with reference to the drawings. However, the scope of the invention is not limited to the illustrated examples. Incidentally, in the following description, the same symbols will be assigned to those with the same function and configuration, and then description thereof will be omitted.

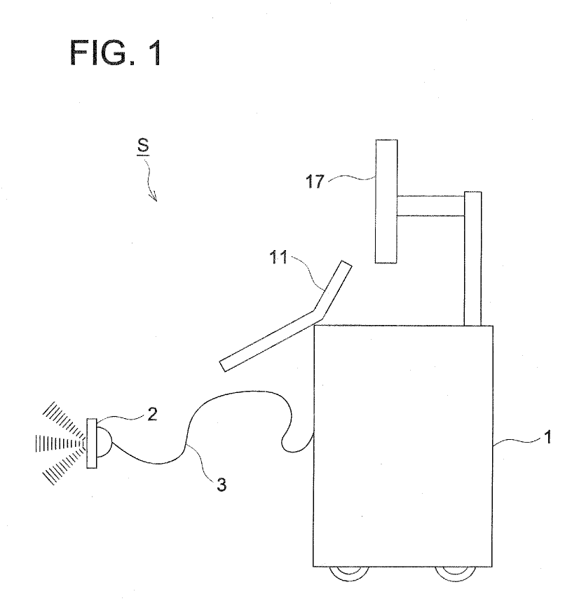

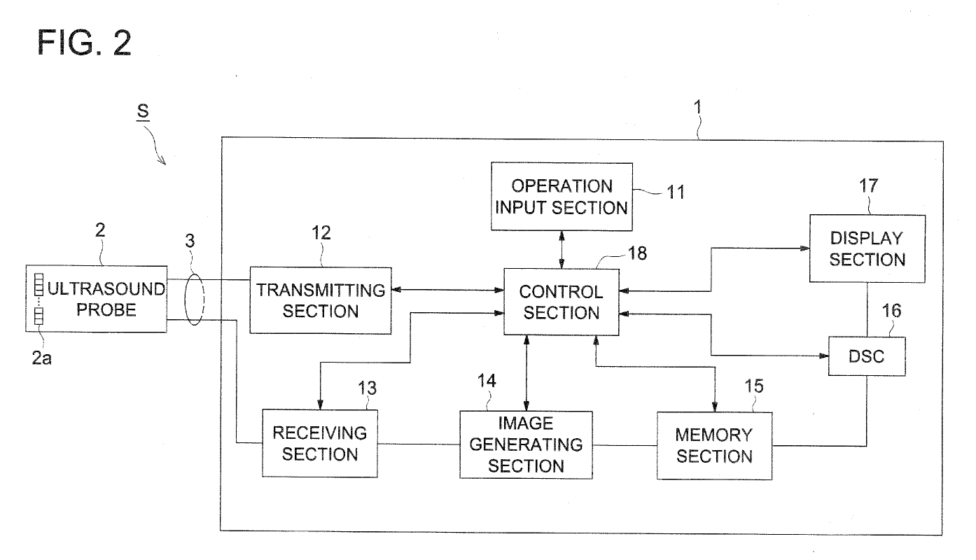

The ultrasound image diagnostic apparatus S according to the present embodiment is provided, as shown in FIG. 1 and FIG. 2, with an ultrasound image diagnostic apparatus main body 1 and an ultrasound probe 2. The ultrasound probe 2 transmits ultrasound (transmission ultrasound) to a subject to be examined (not illustrated) such as a living body and also receives an ultrasound reflection wave (reflected ultrasound: echo) having been reflected by this examined subject The ultrasound image diagnostic apparatus main body 1, connected to the ultrasound probe 2 via a cable 3, transmits a drive signal of an electrical signal to the ultrasound probe 2 to allow the ultrasound probe 2 to transmit transmission ultrasound to a subject to be examined and also to image the internal state of the examined subject as an ultrasound image based on a reception signal of an electrical signal having been generated by the ultrasound probe 2 in response to reflected ultrasound from the interior of the examined subject, which has been received by the ultrasound probe 2.

The ultrasound probe 2 is provided with a transducer 2a containing a piezoelectric element. A plurality of the above transducers 2a are arranged, for example, in a one-dimensional array manner in the orientation direction. In the present embodiment, for example, an ultrasound probe 2 provided with 192 elements of transducer 2a is used. The transducers 2a may be arranged in a two-dimensional array manner. Further, the number of the transducers 2a can be set appropriately. Still further, in the present embodiment, for the ultrasound probe 2, a linear scanning-type electronic scan probe was employed but any of an electronic scanning type and a mechanical scanning type is employable. And, any of a linear scanning type, a sector scanning type, and a convex scanning type may also be employed. Further, in the present embodiment, an ultrasound probe capable of transmitting and receiving ultrasound in a broadband range with excellent sensitivity is effectively employed to acquire a higher-quality ultrasound image. The band width in the ultrasound probe may be appropriately set, but the fractional bandwidth (−20 dB) is preferably at least 100%. It is preferable to employ an ultrasound probe having a transducer capable of receiving a baseband component to be described later together with the fundamental frequency component of reflected ultrasound. The ultrasound probe further preferably has a transducer of a fractional bandwidth of at least 120%, and still further preferably has a transducer of a fractional bandwidth of at least 130%. The fractional bandwidth (−20 dB) is a value obtained by dividing the bandwidth at −20 dB of peak value of the sensitivity by the central frequency.

The ultrasound image diagnostic apparatus main body 1 is configured in such a manner that as shown in FIG. 2, for example, an operation input section 11, a transmitting section 12, a receiving section 13, an image generating section 14, a memory section 15, a DSC (Digital Scan Converter) 16, a display section 17, and a control section 18 are provided.

The operation input section 11 is provided with, for example, various types of switches, buttons, track balls, mouses, and keyboards to input commands to instruct the diagnosis initiation and data such as personal information of an examined subject to output an operation signal to the control section 18.

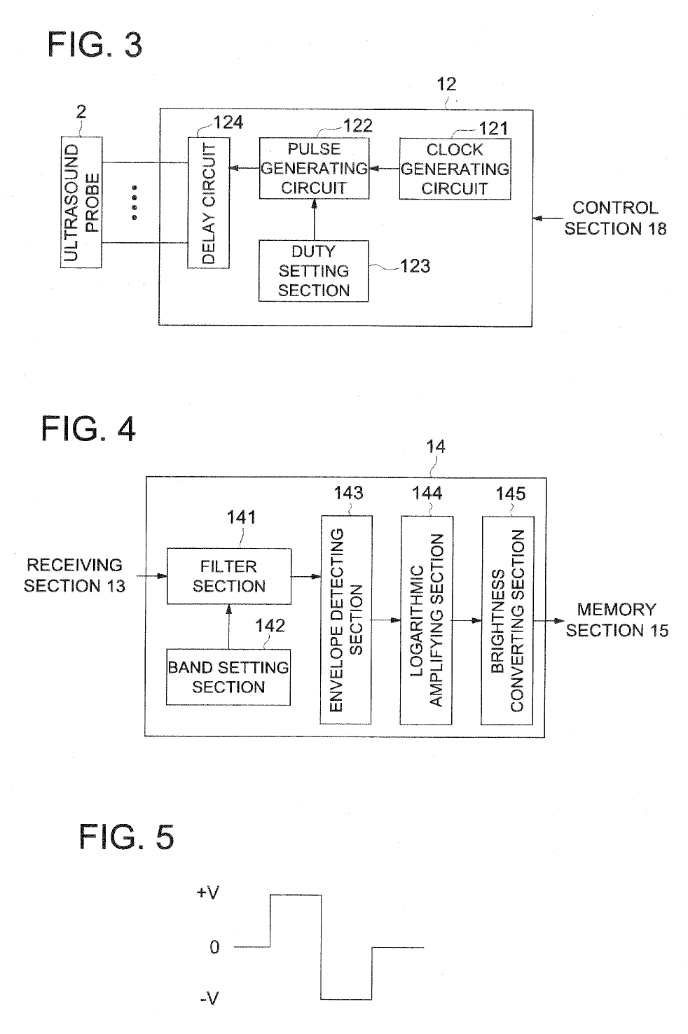

The transmitting section 12 is a circuit, in which in accordance with the control of the control section 18, a drive signal being an electrical signal is fed to the ultrasound probe 2 via the cable 3 to allow the ultrasound probe 2 to generate transmission ultrasound. More specifically, as shown in FIG. 3, the transmitting section 12 is provided with, for example, a clock generating circuit 121, a pulse generating circuit 122, a duty setting section 123, and a delay circuit 124.

The clock generating circuit 121 is a circuit to generate a clock signal to determine the transmission timing and the transmit frequency of a drive signal.

The pulse generating circuit 122 is a circuit to generate pulse signals as drive signals at a predetermined period. As shown in FIG. 5, the pulse generating circuit 122 outputs, for example, 3-valued voltages by switching to generate a square pulse signal. In this case, the amplitude of the pulse signal is set to be identical in positive polarity and negative polarity, which is not restricted to. Thus, employable is a configuration in which binary voltages are switched to generate a pulse signal.

The duty setting circuit 123 sets the duty ratio of a pulse signal output from the pulse generating circuit 122. In other words, the pulse generating circuit 122 outputs a pulse signal having a pulse waveform based on a duty ratio having been set by the duty setting section 123. The duty ratio can be changed, for example, by an input operation using the operation input section 11. Further, employable is a configuration in which an ultrasound probe 2 connected to the ultrasound image diagnostic apparatus main body 1 is identified to set a duty ratio corresponding to the identified ultrasound probe 2.

The delay circuit 124 is a circuit in which with regard to the transmission timing of a drive signal, delay time is set per individual channel corresponding to each transducer and then the transmission of the drive signal is delayed by the thus-set delay time to converge transmission beams containing transmission ultrasound.

The transmitting section 12 configured in the above manner sequentially switches a plurality of transducers 2a to which drive signals are fed by shifting by a predetermined number thereof for every transmission/reception of ultrasound in accordance with the control of the control section 18, and then feeds drive signals to a plurality of the transducers 2a selected for outputting to carry out scanning

The response of a transducer 2a at the time when a pulse signal has been provided to the transducer 2a will be described here.

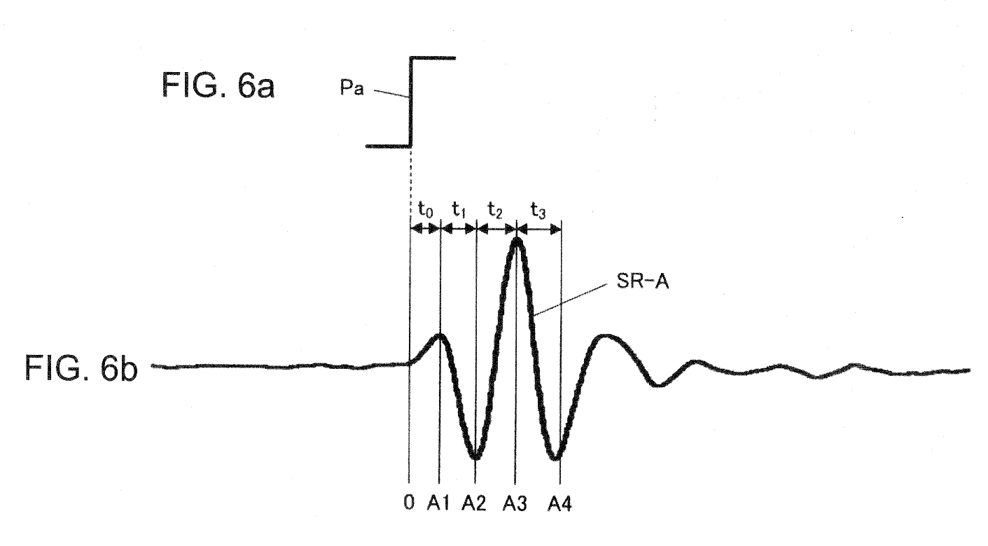

A pulse application Pa, as shown in FIG. 6a, is provided to the transducer 2a from the pulse generating circuit 122 and thereby the transducer 2a returns a step response SR-A as shown in FIG. 6b. This step response is obtained as an integral value in a broadband impulse response. For example, the voltage application time of a pulse application Pa is designated as time 0; the time until a first peak A1 of a step response SR-A from time 0 is designated as t0; the time until a second peak A2 from the first peak A1 is designated as t1; the time until a third peak A3 from the second peak A2 is designated as t2; and the time until a fourth peak A4 from the third peak A3 is designated as t3. As shown in FIG. 6b, when a unipolar voltage is applied to a transducer 2a, the transducer 2a returns a plurality of peaks of amplitude as a response.

A transducer 2a having such response characteristics is allowed to perform a response capable of transmitting high-quality transmission ultrasound by selecting the drive waveform of a pulse signal as described below.

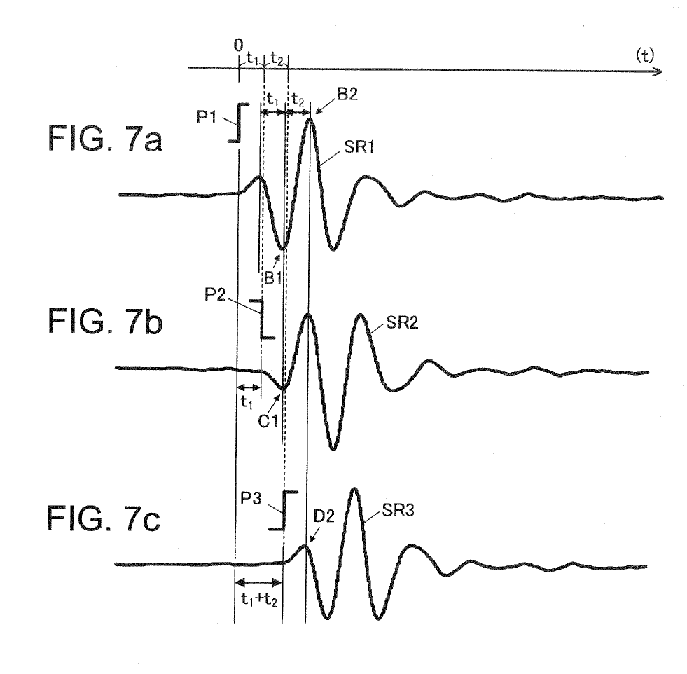

For example, as shown in FIGS. 7a-7c, the case when at a certain time 0, a pulse application P1 as shown in FIG. 7a has been provided is considered. The transducer 2a returns a step response SR1 which is generated by voltage change of the pulse application P1 as shown in FIG. 7a.

Next, in the case when a pulse application P2, as shown in FIG. 7b, whose polarity is reversed with respect to that of the pulse application P1 is provided, a first peak C1 of a step response SR2 which is generated by voltage change of the pulse application P2 and a second peak B1 of the step response SR1 are preferably overlapped to allow the amplitude to increase. Therefore, the application timing of the pulse application P2 is allowed to be delayed by time t1 after the pulse application P1 has been provided. Namely, a first duty of a pulse signal is set at time t1. The time t1 is the time until the second peak from the first peak of the step response SR1 as shown in FIG. 7a. The condition of the overlap between the first peak C1 of the step response SR2 and the second peak B1 of the step response SR1 is satisfied only if the amplitude of the transducer may be increased because of the overlap, and it does not need complete agreement of the positions of the peaks.

Then, in the case when a pulse application P3, whose polarity is different from that of the pulse application P2, that is, the same as of the pulse application P1, is provided, a first peak D2 of a step response SR3 which is generated by voltage change of the pulse application P3 and a third peak B2 of the step response SR1 are preferably overlapped to allow the amplitude to increase. Therefore, the application timing of the pulse application P3 is allowed to be delayed by time t2 after the pulse application P2 has been provided. Namely, a second duty of a pulse signal is set at time t2. The time t2 is the time until the third peak from the second peak of the step response SR1 as shown in FIG. 7a.

In the above manner, the drive waveform of a pulse signal is generated so that one of peaks of a step response generated by one voltage change of the applied pulse overlaps with one of peaks of a step response generated by another voltage change thereafter and thereby the amplitude of the step response of the transducer 2a can be increased and further the pulse width can be decreased. The amplitude of step response of the transducer can be increased effectively by generating the drive waveform of pulse signal so that three peaks constituted of one of peaks of the third step response generated because of voltage change caused thereafter, one of peaks of the second step response and one of peaks of the first step response overlap with one another. The above manner also makes it possible to further increase the amplitude of the step response of the transducer 2a by giving fourth and fifth pulse applications but the pulse width of transmission ultrasound output by this manner is increased by the applications, and the spatial resolution becomes decreased. Therefore, it is preferable to set the drive waveform of a pulse signal in consideration of an appropriate number of times of pulse applications and an appropriate number of peaks of the step response.

Further, the transducer 2a responds so as to cause ringing (tailing) after occurrence of multiple peaks after pulse applications have been provided. However, when a pulse application provided last to return the signal level of a pulse signal to the ground value is allowed to have a time (duty) corresponding to a sufficiently lower frequency or a sufficiently higher frequency than the frequency of the amplitude of the response of the transducer 2a, which is generated via the first and second pulse applications, a low frequency component of ringing is suppressed and then transmission ultrasound having smaller pulse width can be generated. A high frequency component of the ringing is attenuated in the course of outputting transmission ultrasound from the transducer 2a.

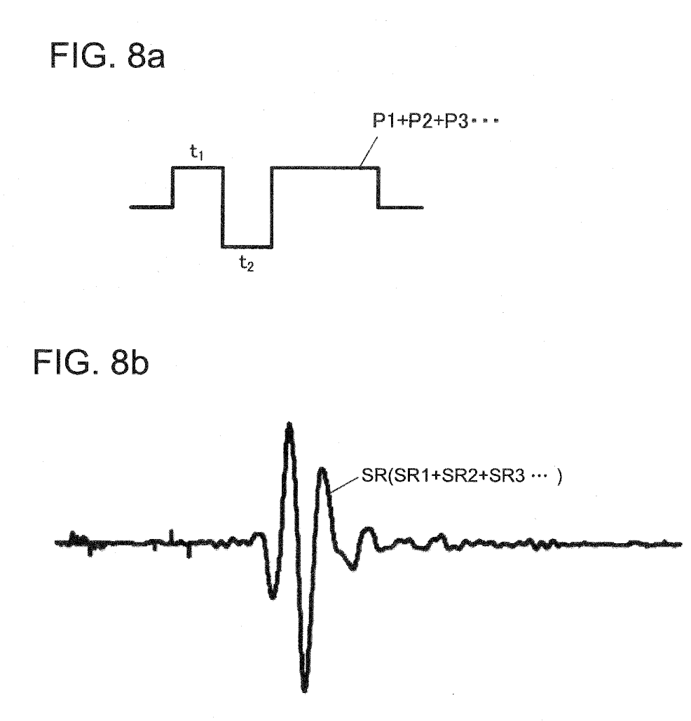

In this manner, the duty ratio of a pulse signal is set so that pulse applications P1, P2, P3, . . . each are provided at the above timing and thereby a pulse signal having a drive waveform being plus-minus asymmetrical as shown in FIG. 8a can be generated. When the thus-generated pulse signal is provided to the transducer 2a, as shown in FIG. 8b, step responses SR1, SR2, SR3, . . . each are added at the above-described timing, and thereby amplitude is largely amplified and also a response SR(SR1+SR2+SR3 . . . ) having small pulse width is obtained.

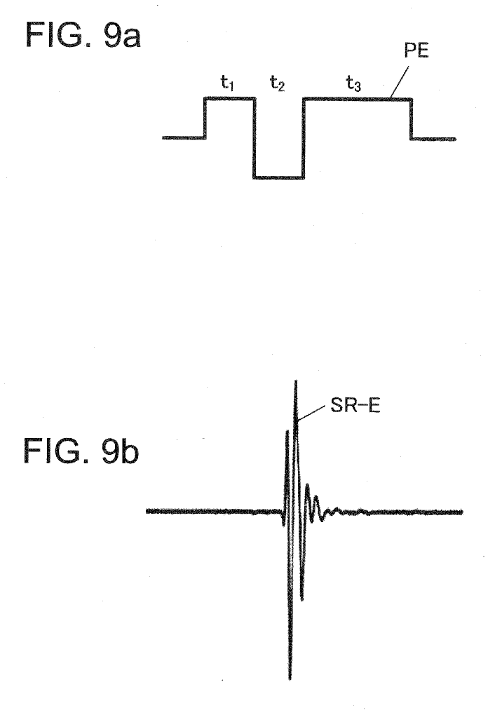

Here, one example of a preferable pulse signal provided to the transducer 2a of the ultrasound probe 2 used in the present embodiment will be described with reference to FIGS. 9a and 9b.

For example, as shown in FIG. 9a, duty ratios in a pulse signal PE output are set so as to gradually increase at an integral multiple ratio of t1:t2:t3=2:7:13. In this case, for example, the times of t1, t2, and t3 as duty are set at 16 ns, 56 ns, and 104 ns respectively. The duty ratios set here are shown as one example which is not restricted to, and it goes without saying that based on the characteristics of a transducer, proper ratios are appropriately set. Further, the duty ratios may be set so as to gradually decrease.

When a pulse signal PE in which its duty ratios have been set with the above ratios is provided to the transducer 2a, as shown in FIG. 9b, a step response SR-E having extremely amplified amplitude with small pulse width is finally obtained.

Conventionally, to improve an ultrasound image in a deep portion, a high-order harmonic component such as a secondary harmonic generated by the non-linearity of tissue in the interior of an examined subject has been utilized to generate ultrasound image data via THI (Tissue Harmonic Imaging)

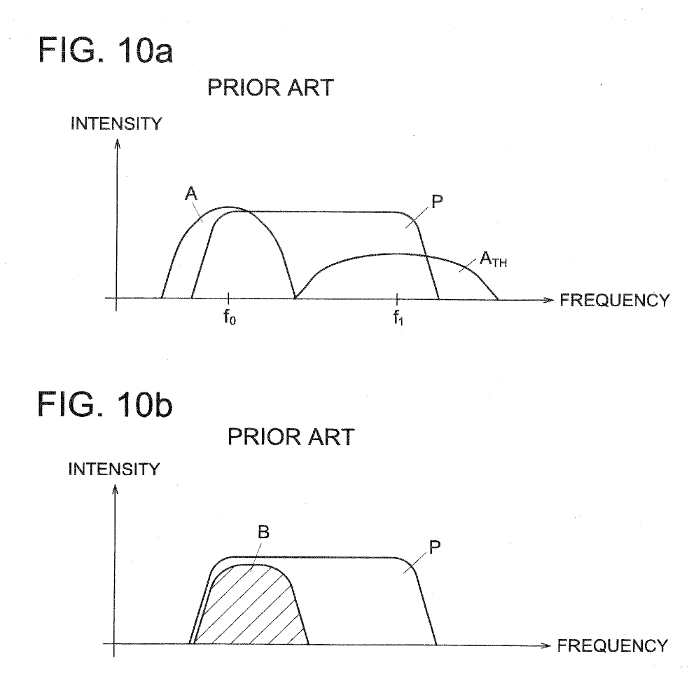

Therefore, for example, as shown in FIG. 10a, even when a broadband transducer having frequency band P is used, a secondary harmonic component A_{TH} having central frequency f₁, twice as large as central frequency f₀ of the fundamental component, needs to be acquired. Therefore, transmission ultrasound having the central frequency f₀ needs to be output. However, as ultrasound to be transmitted has lower frequency, the pulse width increases. Therefore, the frequency band A of transmitted ultrasound is narrowed as shown in FIG. 10a. Further, the frequency of the transmitted ultrasound also decreases and thereby the spatial resolution decreases, although the reaching depth increases.

Further, even when a pulse signal has been provided to a transducer, with respect to a portion departing from the frequency band P of the transducer, in the frequency band A of transmitted ultrasound, the passing is restricted, and thereby the pass frequency band is restricted to a portion shown by B of FIG. 10b.

Due to these causes, conventionally, even when a broadband transducer is used, part of its frequency band is merely used, whereby the characteristics of the transducer have been insufficiently utilized.

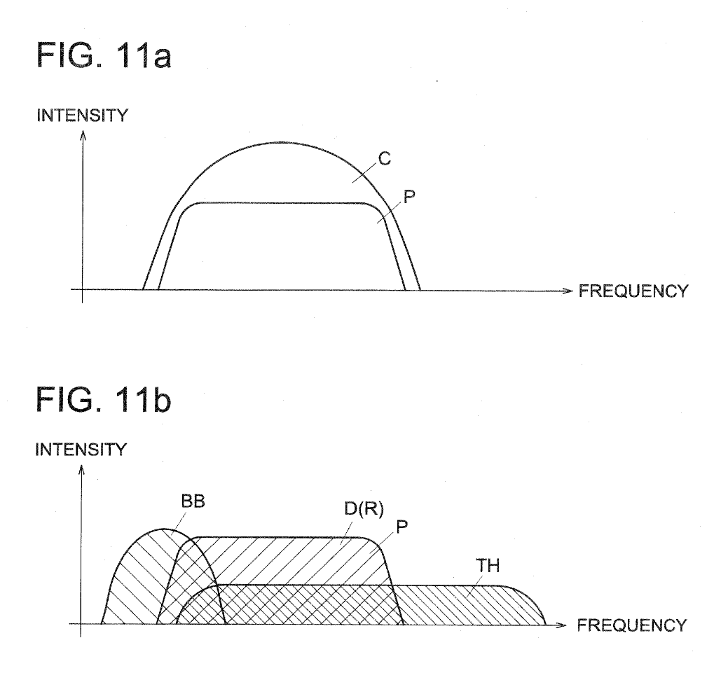

In contrast, when the characteristics of a transducer are taken into account and then, for example, the duty ratio as shown in FIGS. 9a and 9b is set, the frequency band P of a broadband transducer 2a can be provided with a pulse signal having a drive waveform generating transmission ultrasound having wider band characteristics (frequency band C) than the frequency band P of the transducer 2a as shown in FIG. 11a. Such transmission ultrasound having wider band characteristics than the frequency band of a transducer means that the band width of transmission ultrasound is wider than that of a transducer. The band width refers to the frequency range occupying, for example, at the location where intensity attenuates from the peak by 20 dB. The comparison of the band width is carried out by comparing the band width of a transducer and the band width of transmission ultrasound specified above. In this manner, the band width of transmission ultrasound was allowed to be wider than that of the transducer 2a, and thereby as shown in FIG. 11b, in the frequency band C of transmission ultrasound, transmission ultrasound having passed through the band characteristics of the transducer 2a results in one shown by D. Thereby, the frequency band of the transducer is utilized in a wide range and then broadband transmission ultrasound can be output In the present embodiment, as the preferred embodiment, there has been employed a configuration such that the frequency band of the transmit frequency completely covers the frequency band of the transducer 2a but a configuration in which such covering is not completely carried out is employable.

Ultrasound is output as described above and thereby reflected ultrasound containing a broadband high-order harmonic component can be obtained, together with a broadband fundamental component obtained from transmission ultrasound. As shown in FIG. 11b, in this case, the frequency band characteristics of the fundamental component of reflected ultrasound are represented by R, and the frequency band of the high-order harmonic component is represented by TH. In this case, only a portion of the high-order harmonic component TH, which is included in the frequency band P of the transducer 2a passes.

In this manner, in the present embodiment, transmission ultrasound can be output in a broadband range and thereby the frequency band of a transducer can be utilized more widely than conventional. Thereby, a low frequency component and a high frequency component are included and then the compatibility of enhancement of spatial resolution and enhancement of reaching depth can be realized to acquire an excellent ultrasound image.

Further, due to the non-linearity of tissue in the interior of an examined subject, a baseband component is known to be generated. The frequency band of the baseband component depends on the pulse width of reflected ultrasound, regardless of the frequency of transmission ultrasound, and thereby as the pulse width decreases, the frequency of the baseband component increases. In other words, a baseband component can be contained in the frequency band of a transducer depending on the pulse width of reflected ultrasound and the band characteristics of the transducer. Therefore, when this baseband component is utilized and thereby ultrasound image data can be generated, use of the baseband component being a low frequency component makes it possible to acquire an excellent ultrasound image even in a deep portion.

In the present embodiment, since using a transducer 2a having broadband characteristics, transmission ultrasound having small pulse width can be output, as shown in FIG. 11b, the baseband component BB is contained in the frequency band P of the transducer 2a, which, thereby, at least a part of the baseband component BB can pass.

As shown in FIG. 2, the receiving section 13 is a circuit to receive a reception signal being an electrical signal from the ultrasound probe 2 via the cable 3 in accordance with the control of the control section 18. The receiving section 13 is provided with, for example, an amplifier, an A/D converting circuit, a beamforming circuit (a phasing addition circuit). The amplifier is a circuit to amplify a reception signal at a predetermined gain preset for each individual channel corresponding to each transducer 2a. The A/D converting circuit is a circuit to conduct A/D conversion to an amplified reception signal. The beamforming circuit is a circuit, in which an individual channel corresponding to each transducer 2a is provided with delay time for time phase adjustment for an A/D converted reception signal, followed by addition (beamforming) thereof to generate sound-ray data.

The image generating section 14 is provided with, for example, a filter section 141, a band setting section 142, an envelope detecting section 143, a logarithmic amplifying section 144, and a brightness converting section 145.

The filter section 141 is provided with, for example, a band limit filter to filter sound-ray data having been input from the receiving section 13.

The band setting section 142 specifies acquisition depth from the acquisition timing of sound-ray data to output filter characteristic information based on the acquisition depth to the filter section 141.

For example, in the case of filtering a portion at shallow acquisition depth, the band setting section 142 provides filter characteristic information for emphasizing the fundamental component to the filter section 141. The filter section 141, when inputting the filter information to emphasize the fundamental component, suppresses a harmonic component and a noise component contained in sound-ray data to emphasize the fundamental component. Thereby, with regard to a shallow portion of an examined subject, ultrasound image data with enhanced spatial resolution can be generated by the high-frequency fundamental component.

Further, in the case of filtering for a portion at deep acquisition depth, the band setting section 142 provides filter characteristic information to suppress the fundamental component to the filter section 141. The filter section 141, when inputting the filter information to suppress the fundamental component, suppresses the fundamental component contained in the sound-ray data to emphasize a high-order harmonic component such as a secondary harmonic component and/or to emphasize a baseband component Thereby, also with regard to a deep portion of the examined subject, ultrasound image data with enhanced spatial resolution can be generated by the baseband component and/or the high-order harmonic component.

Incidentally, it is also possible that components other than the baseband component are suppressed and the baseband component is emphasized to generate ultrasound image data. Further, it is also possible that ultrasound image data is generated using a fundamental component, a high-order harmonic component, and a baseband component without filtering.

The envelope detecting section 143 carries out full-wave rectification for sound-ray data having been output from the filter section 141 to obtain envelope data.

The logarithmic amplifying section 144 logarithmically amplifies the envelope data.

The brightness converting section 145 carries out amplitude/brightness conversion to quantize the magnitude of a signal shown by the thus-logarithmically amplified envelope data into 256 levels to obtain B-mode image data. Namely, the B-mode image data are data in which the intensity of a reception signal is represented by brightness. Further, the brightness converting section 145 carries out dynamic range and gain adjustments for the envelope data having undergone amplitude/brightness conversion. The B-mode image data having been generated by the image generating section 14 is transmitted to the memory section 15.

As shown in FIG. 2, the memory section 15 contains a semiconductor memory such as, e.g., a DRAM (Dynamic Random Access Memory) and stores B-mode image data having been transmitted from the image generating section 14 on a frame unit basis. Namely, the memory section 15 can store data as ultrasound diagnostic image data configured based on a frame unit basis. Then, the ultrasound diagnostic image data having been thus stored in the memory section 15 is read out in accordance with the control of the control section 18 to be transmitted to the DSC 16.

The DSC 16 converts ultrasound diagnostic image data having been received by the memory section 15 into an image signal based on the scanning system of television signals to be output to the display section 17.

As the display section 17, applicable is a display device such as an LCD (Liquid Crystal Display), a CRT (Cathode-Ray Tube) display, an organic EL (Electronic Luminescence) display, an inorganic EL display, or a plasma display. The display section 17 displays an ultrasound diagnostic image on the display screen in response to an image signal having been output from the DSC 16. Instead of the display device, a printing device such as a printer may be applied.

The control section 18 is constituted of, for example, a CPU (Central Processing Unit), a ROM (Read Only Memory), and a RAM (Random Access Memory), and reads out various types of processing program such as a system program stored in the ROM to be developed on the RAM for central controlling of the operation of each section of the ultrasound image diagnostic apparatus S in accordance with the developed program.

The ROM contains a nonvolatile memory such as a semiconductor and stores a system program corresponding to the ultrasound image diagnostic apparatus S and various types of processing program executable on the system program, as well as various types of data. These programs are stored in the form of a program code which can be read by the computer, and the CPU sequentially executes operations in accordance with the program code.

The RAM forms a work area to temporarily store various types of program executed by the CPU and data relevant to these programs.

As described above, according to the present embodiment, the ultrasound probe 2 has a transducer 2a to output transmission ultrasound toward a subject to be examined using a pulse signal and also to receive reflected ultrasound from the examined subject in order to output a reception signal. The transmitting section 12 outputs a square pulse signal to allow the transducer 2a to generate transmission ultrasound. The transmitting section 12 sets the duty ratio of a pulse signal so that one of peaks of the first step response of the transducer which is generated when a change is made to voltage of the pulse signal overlaps with one of peaks of the second step response of the transducer which is generated when another change is made to the voltage of the pulse signal. As a result, the amplitude of transmission ultrasound can be efficiently amplified and at the same time, pulse width can be decreased, and thereby the transmission ultrasound can be allowed to have a broadband to output transmission ultrasound exhibiting excellent spatial resolution and reaching depth. Further, in the case of use of a broadband ultrasound probe, its band can be efficiently utilized and thereby the fundamental component of reflected ultrasound obtained from transmission ultrasound and a harmonic component due to non-linearity such as a secondary harmonic component and a baseband component generated due to traveling of transmission ultrasound in the interior of an examined subject can be efficiently received. Thereby, a high-quality ultrasound image having excellent spatial resolution and reaching depth can be acquired. Further, since these can be realized by using a square pulse signal, there is no requirement for a complex circuit configuration such as an arbitrary waveform generating system for generating an arbitrary waveform to generate a drive signal having large amplitude in a broadband range or a multiple-valued drive system for switching the voltage of a drive signal in a multiple-valued manner to generate a drive signal having large amplitude in a broadband range, resulting in the possibility of reduced cost in this realization. Further, because energy cost is small, it is excellent for power saving.

Further, according to the present embodiment, the transmitting section 12 sets the duty ratio of a pulse signal so that three peaks constituted of one of peaks of the third step response generated because of voltage change caused thereafter, one of peaks of the second step response and one of peaks of the first step response overlap with one another. Thereby, transmission ultrasound can be efficiently amplified.

Further, according to the present embodiment, the transmitting section 12 outputs a pulse signal such that the frequency band characteristic of transmission ultrasound generated by the transducer 2a is wider than that of the transducer 2a. Thereby, the frequency band of a transducer can be utilized to a maximum extent.

Further, according to the present embodiment, the transmitting section 12 is configured so that the duty ratio of a pulse signal can be changed, and thereby on the basis of the characteristics of an ultrasound probe to be used, the duty ratio of the pulse signal can be optimally set.

Further, according to the present embodiment, the transmitting section 12 changes the voltage of a pulse signal so that the amplitude of the pulse signal is identical in positive polarity and negative polarity, and thereby the circuit configuration is simplified and then cost increase can be minimized.

Further, according to the present embodiment, the filter section 141 filters a predetermined frequency component with respect to a reception signal. The band setting section 142 changes a frequency component to be filtered by the filter section 141 depending on an acquisition depth specified by the acquisition timing of the reception signal. As a result, from a reception signal having been obtained in a broad frequency band, an appropriate frequency component can be extracted based on the depth. For example, with regard to transmission ultrasound having been transmitted in a broadband range, in a shallow depth of about 1 cm, a low frequency component is eliminated so that a high frequency of at least 10 MHz is allowed to be the main component to obtain a higher-resolution ultrasound image, and in a depth of a medium degree to a deeper depth at least 4 cm, a high frequency component is attenuated and therefore, when the upper limit of a frequency component to be filtered is set to be conformed to the frequency component of reflected ultrasound, a signal having higher S/N ratio can be acquired, resulting in acquisition of a high-quality ultrasound image.

Furthermore, according to the present embodiment, since the transducer 2a has frequency band characteristics capable of receiving a baseband component generated due to traveling of transmission ultrasound in the interior of an examined subject, an ultrasound image having excellent spatial resolution even in a deep portion can be acquired.

The description of the embodiment of the present invention is just one example of the ultrasound image diagnostic apparatus according to the present invention and is not limited thereto. The detailed configuration and the detailed operation of each functional section constituting the ultrasound image diagnostic apparatus can be also appropriately modified.

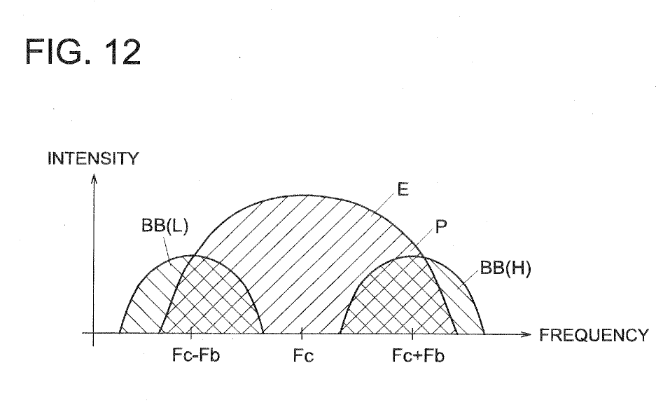

Further, in the present embodiment, the waveform of transmission ultrasound is made to be a waveform which is obtained by laying carrier frequency Fc on baseband frequency Fb and in which the central frequency (carrier frequency) Fc of the transmission ultrasound is higher than the baseband frequency Fb, and then, as shown in FIG. 12, when the range of the frequency band P of the transducer is allowed to contain a frequency obtained by “Fc−Fb” and a frequency obtained by “Fc+Fb”, baseband components BB(L) and BB(H) are contained on both sides of the central frequency of the transmission ultrasound, resulting in acquisition of a higher-quality ultrasound diagnostic image. In FIG. 12, the frequency band characteristic of the transmission ultrasound is shown by E.

According to the embodiment of the present invention, an ultrasound probe having broadband characteristics is efficiently utilized at reduced cost and a high-quality ultrasound image can be acquired.

What is claimed is:

1. An ultrasound image diagnostic apparatus comprising:
an ultrasound probe which has a transducer for outputting a transmission ultrasound toward a subject to be examined by using a pulse signal and for receiving a reflected ultrasound from the subject so as to output a reception signal; and
a transmitting section for outputting a square pulse signal so as to allow the transducer to generate the transmission ultrasound,
wherein the transmitting section is configured to set a duty ratio of the pulse signal so that one of peaks of a first step response of the transducer overlaps with one of peaks of a second step response of the transducer, the first step response being generated when a change is made to voltage of the pulse signal and the second step response being generated when another change is made to the voltage of the pulse signal.

2. The ultrasound image diagnostic apparatus of claim 1,
wherein the transmitting section is configured to set the duty ratio so that three peaks constituted of one of peaks of a third step response, one of peaks of the second step response and one of peaks of the first step response overlap with one another, the third step response being generated because of voltage change caused after the first and the second step responses.

3. The ultrasound image diagnostic apparatus of claim 1,
wherein the transmitting section is configured to output the pulse signal so as to ensure that a frequency band characteristic of the transmission ultrasound generated by the transducer is wider than a frequency band characteristic of the transducer.

4. The ultrasound image diagnostic apparatus of claim 1,
wherein the transmitting section is configured to be capable of changing the duty ratio.

5. The ultrasound image diagnostic apparatus of claim 1,
wherein the transmitting section is configured to change the voltage of the pulse signal so that an amplitude of the pulse signal is identical in positive polarity and negative polarity.

6. The ultrasound image diagnostic apparatus of claim 1, further comprising:
a filter section for filtering a predetermined frequency component in the reception signal; and
a band setting section for changing a frequency component to be filtered by the filter section according to an acquisition depth specified by time of acquisition of the reception signal.

7. The ultrasound image diagnostic apparatus of claim 1,
wherein the transducer is configured to have a frequency band characteristic which enables the transducer to receive a baseband component generated due to traveling of the transmission ultrasound in an interior of the subject.
